(19) United States
(12) Patent Application Publication
(10) Pub. No.: US 2006/0155305 A1
(43) Pub. Date: Jul. 13, 2006

(54) Extraction device

(76) Inventors: Franz Freudenthal, La Prez (DE); Rolf W. Gunther, Aachen (DE); Thomas Schmitz-Rode, Aachen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/527,390

(22) PCT Filed: Sep. 11, 2003
(86) PCT No.: PCT/EP03/10116

§ 371 Date: Oct. 17, 2005

(30) Foreign Application Priority Data
Sep. 11, 2002 (DE) 102 42 444.6

(51) Int. Cl. A61B 17/26

(52) U.S. Cl. 606/114

(57) ABSTRACT

In an extraction device (1) for extracting objects (2), in particular clots, foreign bodies, etc., from cavities (3) in a human or animal body, with first and second compressible and expandable collecting baskets (10, 20, 102, 110) between which the object (2) can be captured, said collecting baskets being mutually displaceable and being able to be drawn one into the other, at least one collecting basket (10) is umbrella-like in the expanded state and is designed with flexible wire-like adjustment elements for deliberately changing the shape and/or position of the collecting basket so that the object can be captured in the latter and can be drawn into the other collecting basket (20).

The invention relates to an extraction device for extracting objects, in particular clots, foreign bodies, etc., from cavities in a human or animal body, with first and second compressible and expandable collecting baskets between which the object can be captured, which collecting baskets are mutually displaceable and can be drawn one into the other.

Even in the era of prophylactic anticoagulation, acute pulmonary embolism represents a frequent and often life-threatening event. In cases of massive pulmonary embolism, standard treatment consists of medicinal thrombolysis with streptokinase, urokinase or tissue plasminogen activator. The objective of this is the recanalization of the affected vessel. After these standard treatments have been applied, monitoring by imaging processes such as echocardiography, angiography or computed tomography, shows only slight recanalization after thrombolysis. Despite high-dose thrombolysis, therefore, some patients die of right heart failure.

Alternatively, if thrombolysis is contraindicated, for example in cases of intracranial injury or after operations, or if thrombolsysis has failed, the embolus material can be removed surgically. The intervention using a heart-lung machine places a considerable physical burden on the patient and is associated with a high mortality rate.

Therefore, various devices have been developed to make clearance and recanalization of the vessels easier.

In 1964, Greenfield developed a mechanical instrument with endoscope-like control which extracts pulmonary emboli non-surgically via a venous puncture. Because of its complicated handling, this suction catheter did not gain widespread acceptance. In 1991, Gunther and Schmitz-Rode developed a high-speed catheter system for fragmentation of pulmonary emboli which, because of its technical complexity and the insufficient pulmonary control of the catheter, also failed to gain widespread acceptance. A modified pigtail catheter developed by Gunther and Schmitz-Rode in 1995 is moved in rotation in the embolic occlusion and in this way effects coarse fragmentation of the embolus material. However, only the soft and fresh embolus material can be removed in this way. The modified pigtail catheter fails in cases where there are more solid, organized emboli. In the case of pulmonary arteries filled centrally to peripherally with embolus material, there is little prospect of successful recanalization by fragmentation, since the fragments cannot float off toward the periphery.

These disadvantages do not arise in US 2002/0095161 A1. In this device for extracting stones from the ureter, for example, these stones are captured in a basket having a large opening which covers over half of the surface of the basket and through which stones and stone fragments can pass into the interior of the basket. In addition, the basket has relatively narrow openings which are.suitable for holding back the stones and stone fragments. A disadvantage of this has proven to be that, despite the possibility of rotating the basket, the capturing operation is relatively awkward and the stone to be captured does not pass into the basket without any problems.

A similar problem arises in the extraction device according to U.S. Pat. No. 5,779,716 in which a sack-like collecting basket is provided with a wire at its proximal end, which wire keeps the proximal opening of the sack-like collecting basket open in order to assist in the capturing operation.

The prior art also includes extraction devices with coiled wires between which a stone or other foreign body can be captured. An example of this is set out in WO 99/47054. There is a risk here of the stone escaping from the loops while being drawn back in the recovery operation. This applies also to the extraction device disclosed in WO 01/05311 A1 and to the extraction device disclosed in US 2002/0026203 A1.

Gripper-like means which take hold of the stone to be recovered and secure it during the recovery operation are likewise known in the prior art, for example from WO 00/54672 A1.

To remove clots from the vessel system, U.S. Pat. No. 5,419,774 A discloses an extraction device whose distal end is provided with a chamber into which the clot is drawn by suction. Situated in the chamber there is a separating device which separates that part of the clot that is situated in the chamber. A pressurized fluid is delivered in order to carry off the clot and the blood collecting with the latter. The structure of the device, however, is relatively complicated since, on the one hand, a means for applying a partial vacuum has to be provided and, on the other hand, a means for delivering a pressurized fluid. Moreover, a means has to be provided for separating the clot. These disadvantages also apply to the catheter for working on and removing soft and hard substances for use in invasive microsurgery and vessel treatment according to DE 197 34 890 C1. The area of application of the latter is the removal of tissue or gallstones, for example.

For removal of clots, it is also known in the prior art, from U.S. 2002/0026211 A1, to provide a device and a method for filtering of emboli or removal of clots from a vessel, in which the device has a vascular filter for trapping the emboli and optionally a thrombectomy element for removal of the clot. The vascular filter contains a support ring with one or more hinge areas which are secured near a distal end of a guide wire, and a blood-permeable pouch which is secured on the support ring. The support ring forms the opening of the blood-permeable pouch and holds it open. It is possible to provide two pouches one behind the other, their openings pointing in the same direction, namely in the proximral direction. The first vascular filter captures the clot, the second one the remaining emboli. Both vascular filters are retracted into a tube or catheter together with the clot or the emboli. This device proves disadvantageous on account of the support rings in conjunction with the blood-permeable pouch since the hinges provided in the support rings make it expensive to produce. In addition, because of the use of a support ring, the risk of damaging the vessel from which the clot or emboli are to be removed is very high, since this support ring is rigid and immovable compared to the vessel and may scrape against the inside wall of the vessel.

A further device for the removal of clots is known from U.S. Pat. No. 5,011,488 A. Here, a vascular catheter system comprises an outer flexible tube, an inner flexible tube disposed in the lumen of the outer flexible tube, and an expandable body mounted at the distal end of a third flexible tube itself disposed in the lumen of the inner flexible tube. The inner tube includes an expandable tip which can open to adopt substantially the cross section of a blood vessel. The expandable body is positioned in the clot region and expanded. The clot material to be removed is then situated between the two expanded parts, the expanded tip being an inflatable body which bears against the inside wall of the vessel and, when drawn back into the tube, scrapes the clot material off from the inside wall of the vessel and carries it toward the expanded body. The inflatable body or balloon fits into the expanded open body and has a truncated cone shape corresponding to the latter. The expanded body has a plurality of spring elements which, after being pushed out of the tube, lead to opening of the expandable tip. Because of the truncated cone shape, the expanded body is pushed together again when drawn back into the tube. The same happens to the inflatable body or balloon then lying on the inside. This device proves disadvantageous since, when the two bodies are drawn back, the clot material located between them can escape again, especially when the inflatable body is compressed, i.e. it is not held securely between the inflatable body and the expandable open truncated cone shaped body.

WO 00/51505 A1 discloses an extraction device with only a distal portion which can be widened like a balloon and has intertwined wires covered by a woven fabric. To clear a vessel, the widened end scrapes along the vessel wall and the woven fabric prevents penetration of scraped-off foreign bodies into the device or into the widened portion.

DE 692 28 326 T2 discloses an extraction device in which a flexible loop part is covered with a net and secured at its distal end and proximal end on a tensioning wire. By moving the loop part along the tensioning wire, it is possible for the net to assume an open form and a closed form. In this way, a foreign body can be captured in the loop part covered with mesh.

WO 00/53120 A1 discloses an extraction device in which two collecting baskets are provided with the openings of the collecting baskets directed toward one another. The distal collecting basket is secured on a rod and is arranged inside the other collecting basket so that it can be drawn inside the latter. The distal collecting basket has a distal contracted end, whereas the proximal collecting basket has a proximal contracted end.

The object of the present invention is to avoid the aforementioned disadvantages of the prior art and create an extraction device for extracting objects, in particular clots, foreign bodies, etc., from cavities in a human or animal body, in which device the object is securely covered so that it can be safely recovered and then removed from the cavity of the human or animal body. In particular, the aim is to permit effective and non-surgical recanalization in the case of partially organized emboli and in cases of extensive embolization of the pulmonary circulation.

The object is achieved with an extraction device in accordance with the preamble of claim 1 in which at least one collecting basket is umbrella-like in the expanded state and is designed with flexible wire-like adjustment elements for deliberately changing the shape and/or position of the collecting basket so that the object can be captured in the latter and can be drawn into the other collecting basket. For an extraction device in accordance with the preamble of claim 5, the object is also achieved by the fact that at least one wire-like flexible adjustment element is secured on the distal and/or proximal end in such a way that the at least one collecting basket can be deliberately controlled and changed in shape. Developments of the invention are defined in the dependent claims.

Thus, an extraction device for extracting objects, in particular clots, foreign bodies, etc., from cavities in a human or animal body is created which makes it possible in particular for emboli, in particular in pulmonary arteries, to be captured, covered and compressed so that they can be safely recovered from the circulatory system by way of the right heart and removed via the percutaneous venous access either by the femoral route or by the jugular route. Using at least one umbrella-like collecting basket which can be drawn into the other collecting basket means that it is possible for clot particles to be squeezed in the collecting baskets so that the large percentage of fluid present in the clot can escape, and thus only small clot particles are carried off with this fluid into the blood circulation system, so that this no longer poses a life-threatening risk. The larger clot particles, by contrast, are drawn through a channel element from the human or animal body so that they too no longer represent a threat to life. The extraction device can therefore be used in particular as a thrombectomy system, making it possible in particular to avoid massive pulmonary embolism. Vascular deposits can be taken up and removed, this being possible in particular also for the vessels leading in the direction of the brain. Another area of application is the removal of foreign bodies, which also includes for example the removal of kidney stones, gallstones and other bodies forming in the organism. Moreover, “real” foreign bodies can be removed, for example from the lung system, from the gastrointestinal system and generally from all the cavities of a human or animal body. The extraction device is thus designed not only for use in all vessel regions, for example in miniaturized form for thrombectomy in hemodialysis shunts, in minimally invasive surgery and robotic surgical interventions, but generally for removal of all kinds of foreign bodies or bodies from cavities of a human or animal body, for example also by laparoscopy.

By providing the at least one collecting basket with an umbrella shape having flexible wire-like adjustment elements, it is possible to obtain any desired adjustment in size and thus any change in the collecting range. The umbrella-like collecting basket preferably opens toward the other collecting basket. In this way, the object, for example clot, foreign body, etc., can be captured between the two collecting baskets oriented with their openings toward one another and thus collected. “Escape” of the object is no longer possible. Alternatively, the umbrella-like collecting basket can open in the direction pointing away from the other collecting basket. This possibility proves advantageous if the one collecting basket cannot be guided past the object to be recovered and take hold of said object from the other side. Therefore, both collecting bodies are arranged on the same side, namely on the proximal side, of the object, and the umbrella-like collecting basket engages the object from this proximal side. However, the latter is then likewise drawn into the other collecting basket in order to recover it and if possible compress it.

Both collecting baskets are preferably provided with at least one adjustment element for adjusting the shape and/or position of the collecting baskets. The at least one adjustment element is used to move the collecting basket forward and backward in the cavity from which the object, e.g. clot, foreign body, etc., is to be removed and for adjusting the shape and size of the collecting basket so as to permit effective adaptation to the object and to the latter's position. The at least one adjustment element preferably has one or more thin wires. These wires permit particularly good and exact change of shape of the collecting basket. In at least one collecting basket, actuation of the adjustment elements can lead to widening of the collecting basket. This proves especially advantageous in the collecting basket into which the other collecting basket is to be drawn. The other umbrella-like collecting basket is advantageously also provided with adjustment elements in order to position it around the clot, foreign body, etc., in such a way that the latter can be safely captured. This crucially distinguishes the collecting baskets according to the invention from those disclosed in US 2002/0095161 A1, since the latter discloses only a large distal collecting basket without adjustment elements as advantageous. With likewise preferred provision of a guide cannula secured at the distal end of the collecting basket or of the first collecting basket, the width of opening in particular of this first collecting basket can be altered if proximal adjustment elements are provided. In this case, the collecting basket can be kept at the optimum location by the guide cannula and can be opened as far as is necessary by the adjustment elements. In the case of a channel element which is likewise preferably provided and through which the collecting bodies can be conveyed to the site where the object, e.g. clot, foreign body, etc., lies in the hollow cavity, the collecting basket can be drawn back together with the guide cannula and the adjustment elements, with compression of the collecting basket or collecting baskets taking place.

The at least one adjustment element is preferably arranged on the outside and/or inside of the at least one collecting basket. The at least one adjustment element is particularly preferably integrated at least partially into the circumferential surface of the collecting basket and/or laced into it. This proves especially advantageous in the second collecting basket which receives the first collecting basket, so as to be able to bring it into an optimal shape in a deliberate and reproducible manner. The proximal end of the collecting basket is preferably secured in a tubular element, in particular a catheter, and the at least one adjustment element is routed through the tubular element. By applying a force in the distal or proximal direction, the collecting basket can be adjusted in shape. The collecting basket is preferably designed in such a way that it shortens in its longitudinal direction when it is widened, and lengthens when its cross section is reduced. The at least one collecting basket can preferably be expanded to a diameter greater than the diameter of the cavity, in particular vessel, to be cleared, so as to permit partial widening of said vessel. This advantageous possibility of shortening of its length means that, at the same time as the vessel widens and upon positioning in front of an object to be captured, this object can automatically spring into the collecting basket. In the stents known from the prior art, such shortening is absolutely not wanted for bringing foreign bodies into them in a particularly effective manner. In the context of the present invention, however, the change in cross section and the shortening of the length of the collecting basket prove especially advantageous for the collecting procedure.

To strengthen the connection between tubular element and collecting basket, a sleeve element can preferably be provided at the proximal end of the collecting basket. This sleeve element, however, is advantageously applied in such a way that adjustment in particular of the shape of the collecting basket is easily possible via the adjustment elements.

The at least one adjustment element preferably protrudes beyond the outstretched length of the at least one collecting basket and is preferably arranged to be actuated from the proximal end. To actuate the adjustment element or elements, a handgrip is preferably provided, in which case the adjustment elements of all the collecting baskets can preferably be actuated via one handgrip. The handgrip for this purpose preferably has different actuating elements to which the individual adjustment elements can be secured.

The adjustment element or elements are preferably secured on the collecting basket in a branched-out configuration and are brought together in groups proximally. This proves especially advantageous on the umbrella-like collecting basket so that the latter can be guided especially effectively around the object, for example clot, foreign body, etc., and can be drawn back smoothly to the channel element or second collecting basket. The at least one adjustment element is preferably made in one piece with the collecting basket. In this way, there is no risk of the adjustment elements coming loose from the collecting basket. Moreover, the actuating force can continue optimally through the circumferential surface of the collecting basket, so that the actuation of the at least one adjustment element as soon as possible provides the desired effect of adjustment of the collecting basket.

The distance between the distal end of the collecting basket and the at least one proximal point of attachment or point of emergence of the at least one adjustment element remains constant for different designs of the collecting basket. This proves advantageous upon introduction into the channel element because there is no risk of losing the captured foreign body, clot, etc. Staggering of the movement of insertion into the element can also be avoided by this means.

Reducing elements are preferably provided transversely with respect to the longitudinal extent of the at least one collecting basket, in particular in the area of the proximal and/or distal ends of the collecting basket and/or in the area of the at least one proximal point of attachment or point of emergence of the at least one adjustment element. The reducing elements are particularly preferably nooses. In this way, before insertion into the channel element or a catheter, the at least one collecting basket can be closed so that the object is trapped completely in the collecting basket and cannot accidentally escape back into the cavity, in particular into a blood vessel. This proves especially advantageous for the umbrella-like collecting basket.

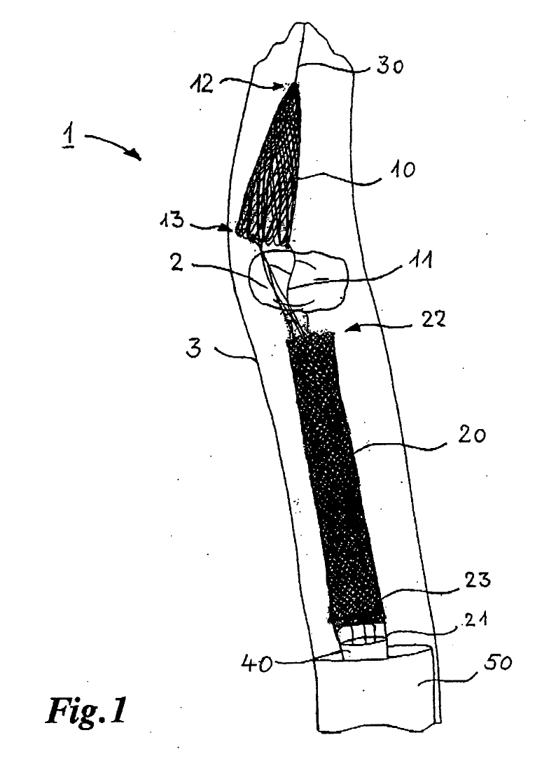

At least one collecting basket is preferably composed of a braided fabric and/or woven fabric and/or scrim, in particular a wire braid and/or woven wire fabric and/or wire scrim. The at least one adjustment element is particularly preferably made of part of the braided fabric, woven fabric or scrim. When a collecting basket made of such a braided fabric, woven fabric or scrim is provided, the mesh size can be used to determine the size of the particles which, after compression of the collecting basket, are introduced into for example the channel element or a catheter and back into the blood stream or generally the cavity of the human or animal body. Squeezing or pressing out of, in particular a clot or embolus, is thus possible in a particularly effective manner. Alternatively, at least one collecting basket can be made up of a tube which is slotted at least along part of its length. In this respect, reference is made in particular to DE 100 00 137 A1. The elements shown in particular in FIG. 1 of the latter document can advantageously also be used here as collecting bodies. It proves especially advantageous if the cut or cuts in the slotted tube are made in such a way as to afford the maximum ratio of shortening and widening upon expansion of the collecting basket in particular secured at the proximal end. It is particularly preferable for the cut(s) in the slotted tube to be made long by comparison with the longitudinal extent of the collecting basket. In this way, it is possible to achieve especially good widening and shortening during expansion of the collecting basket.

A guide wire or inner mandrel is preferably provided along which the at least one collecting basket or the two collecting baskets can move and/or can be introduced into the cavity. In this way, it is possible to provide an optimal orientation with respect to the object to be captured (clot, foreign body, etc.), particularly upon withdrawal into the channel element or a catheter.

At least the second collecting basket preferably has a self-opening partial area and a self-closing partial area, which self-closing partial area can be opened deliberately by at least one adjustment element. In this way, the object captured is retained particularly securely and effectively.

The extraction device can preferably also be used in conjunction with an endoscope with or without the provision of the channel element. In this way, the capturing and extracting procedure can be monitored particularly clearly from outside, since it is possible not just to advance a viewing optic, but also a light source, to the operating site.

Partial areas of the at least one collecting basket preferably are made of material with differing diameter. It is particularly preferable that an expandable partial area of the at least one collecting basket is made of a material with a thinner cross section or preferably has a braided fabric, scrim or woven fabric with filaments of different diameter and/or cross section. In this way, a partial area of the collecting basket can be made particularly easy to expand, whereas the partial area not to be expanded, which is secured in particular on the tubular element or catheter, can be made of material with a thicker cross section or uniform cross section. This provides the advantageous possibility of designing the collecting basket all across its circumferential surface in such a way that certain areas can be particularly easily expanded so as to permit the best possible collecting and extracting procedure. The material of the at least one collecting basket is particularly preferably treated chemically and/or mechanically in at least a partial area, in particular etched, electrolytically polished, microground or otherwise treated. In addition, or alternatively, a partial area can by this means be provided with expansion and compression properties different than those of the remaining areas of the collecting basket.

The at least one collecting basket is preferably made of a biocompatible material, in particular a metal or a metal alloy, in particular a stainless steel or nitinol. Partial areas of the at least one collecting basket can also be made of different materials, in particular ones with different mechanical properties. It is thus possible to provide filaments not just of different diameter, but also of different elasticity, flexibility and/or mechanical loading capacity.

The guide cannula and/or the tubular element(s) are preferably made of a flexible material, in particular a metal, a metal alloy, a plastic or another flexible material or a combination of materials, in particular nitinol. Depending on the site of use and type of use, it proves helpful if the guide cannula and/or the tubular element(s) can simulate the bends of a blood vessel or of another vessel or cavity into which they are introduced. Depending on the location from which the extraction device is introduced into the patient's body, a greater or lesser number of bends have to be simulated. Therefore, the channel element is also advantageously made of a stable and at least partially flexible material, in particular of a plastic, metal, a metal alloy, in particular nitinol, in particular a thin-walled nitinol tube. However, it is preferably configured in such a way that there is no risk of injury to the vessel or other cavity into which the channel element is introduced. The adjustment elements can on the one hand be part of the braided fabric, woven fabric or scrim or also part of the slotted tube of the collecting basket. The provision of a slotted tube in the form of a collecting basket proves advantageous especially in terms of the reproducibility of the shape since, with laser cutting of the tubes, machine automation leads to a considerable reduction in production costs. Therefore, the adjustment elements are advantageously made of the same material as the at least one collecting basket or the collecting baskets. The tubular element too can therefore preferably be designed in one piece with the second collecting basket and be provided at least partially with a cut permitting expansion and compression.

The invention is explained in greater detail below on the basis of illustrative embodiments and with reference to the drawings, in which:

FIG. 1 shows a plan view of a first embodiment of an extraction device according to the invention,

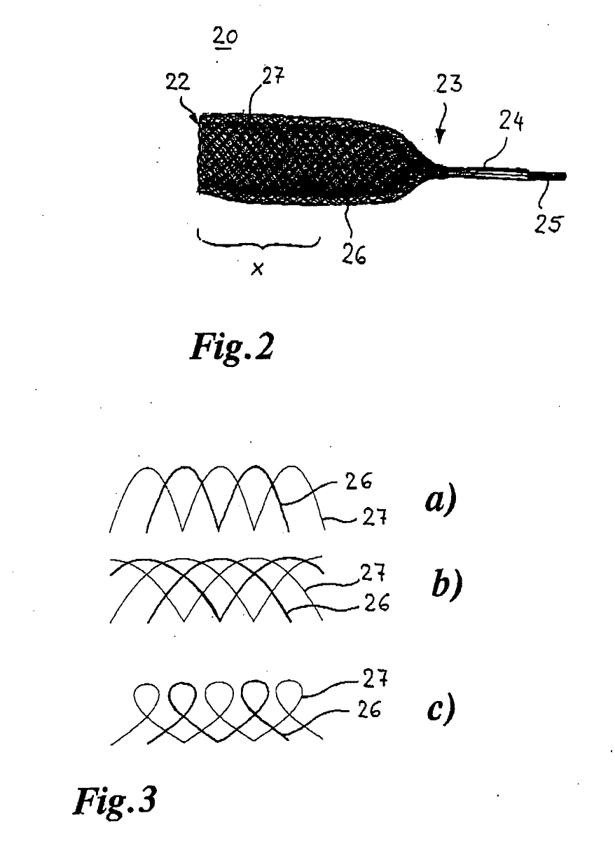

FIG. 2 shows a plan view of an alternative embodiment of a second collecting basket according to the invention for an extraction device according to FIG. 1,

FIGS. 3a) through c) show sketches of three end areas of collecting baskets designed according to the invention,

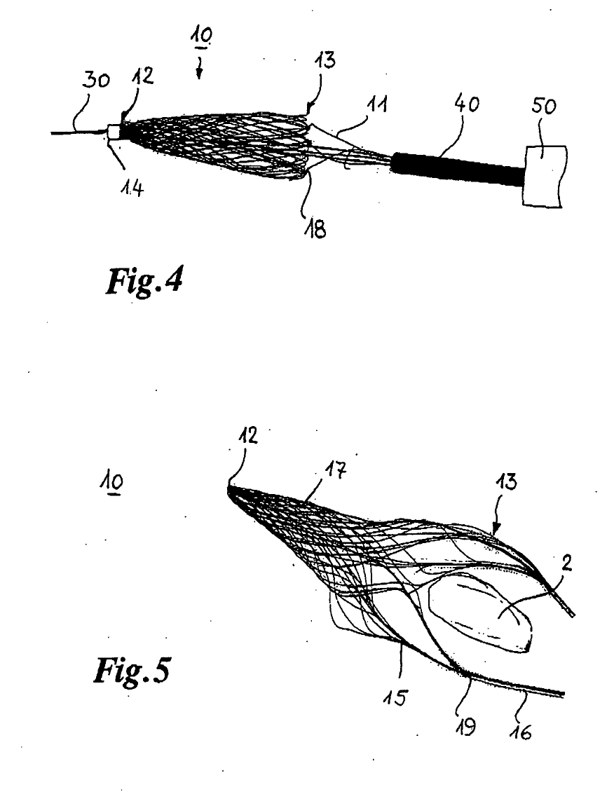

FIG. 4 shows a plan view of a first collecting basket designed according to the invention with guide sleeve, guide wire, adjustment elements, catheter and channel element,

FIG. 5 shows a plan view of a second embodiment of the first collecting basket, with the object to be captured,

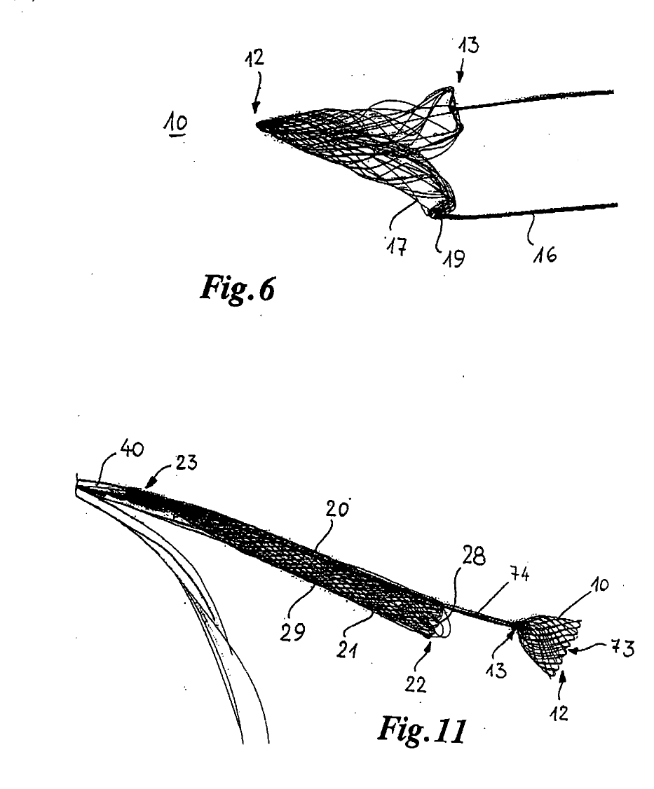

FIG. 6 shows a plan view of a third embodiment of a first collecting basket according to the invention,

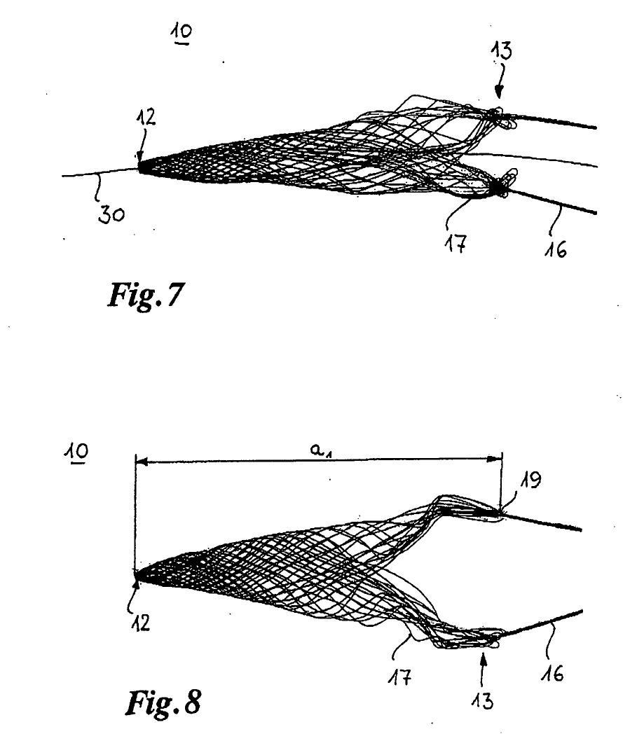

FIG. 7 shows a plan view of a fourth embodiment of a first collecting basket according to the invention,

FIG. 8 shows a plan view of a fifth embodiment of a first collecting basket designed according to the invention,

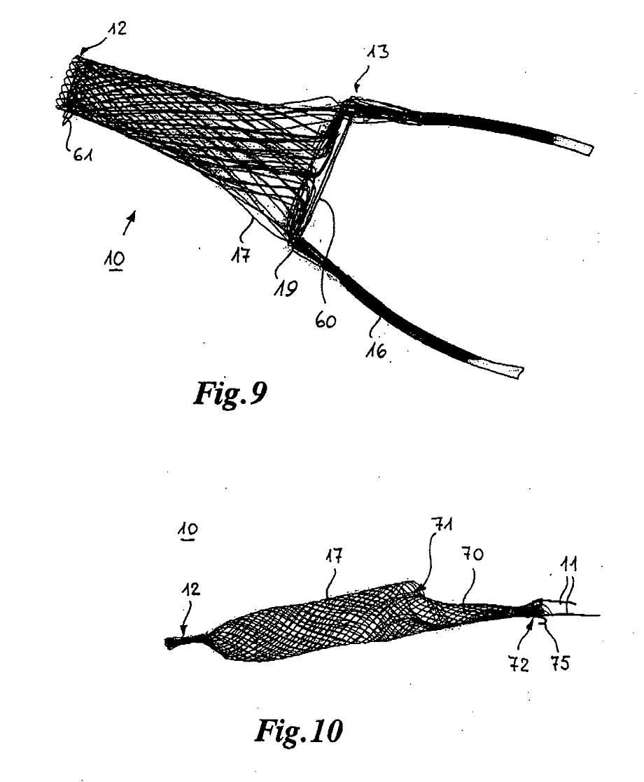

FIG. 9 shows a plan view of a sixth embodiment of a collecting basket designed according to the invention and with reducing elements,

FIG. 10 shows a plan view of a seventh embodiment of a first collecting basket,

FIG. 11 shows a plan view of an alternative embodiment of an extraction device according to the invention,

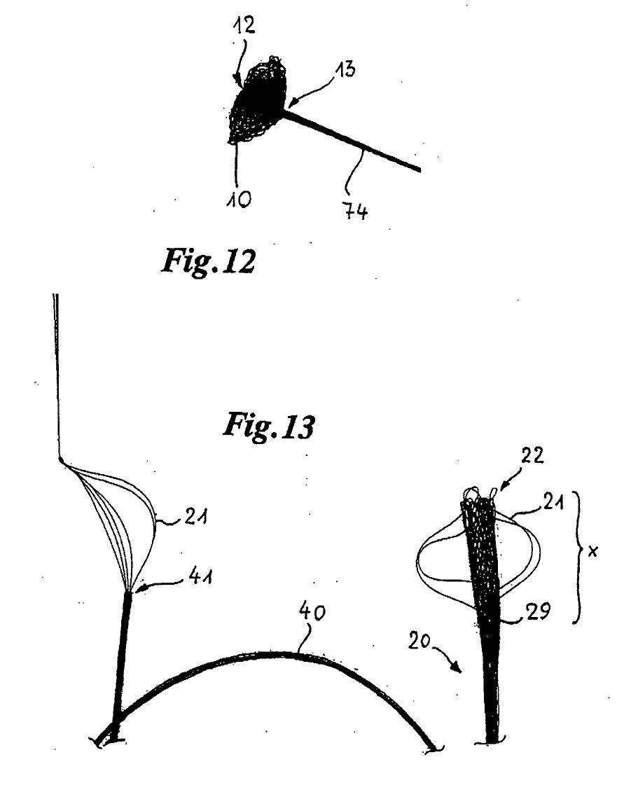

FIG. 12 shows a plan view of a further alternative embodiment of a first collecting basket according to the invention,

FIG. 13 shows a plan view of a second collecting basket with adjustment elements, in the compressed state,

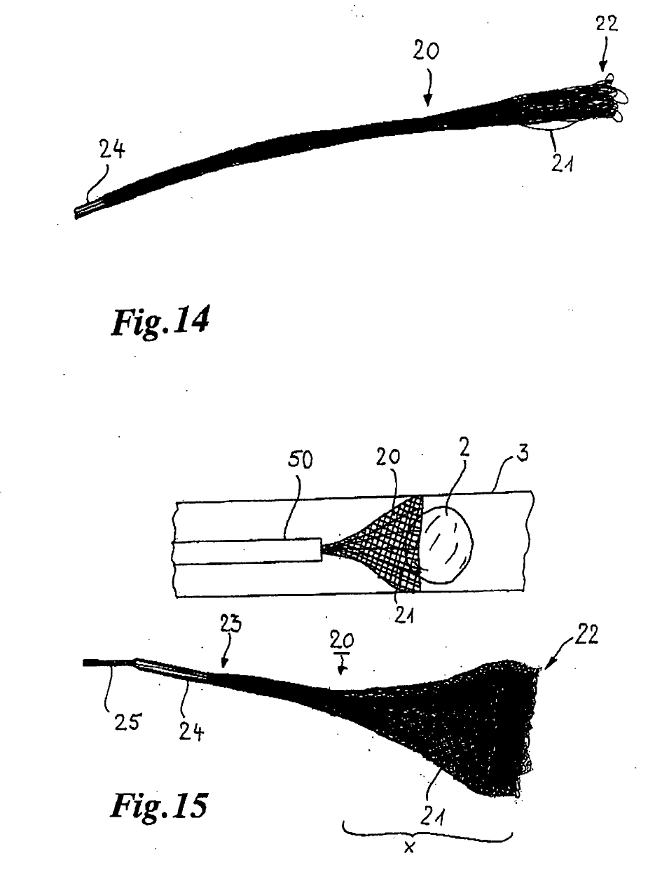

FIG. 14 through FIG. 19 show individual steps in the collecting procedure using the second collecting basket according to the present invention,

FIGS. 20a) and b) show a sketch of a further embodiment of a collecting basket according to the invention, cut from a slotted tube, in the compressed state (a) and expanded state (b),

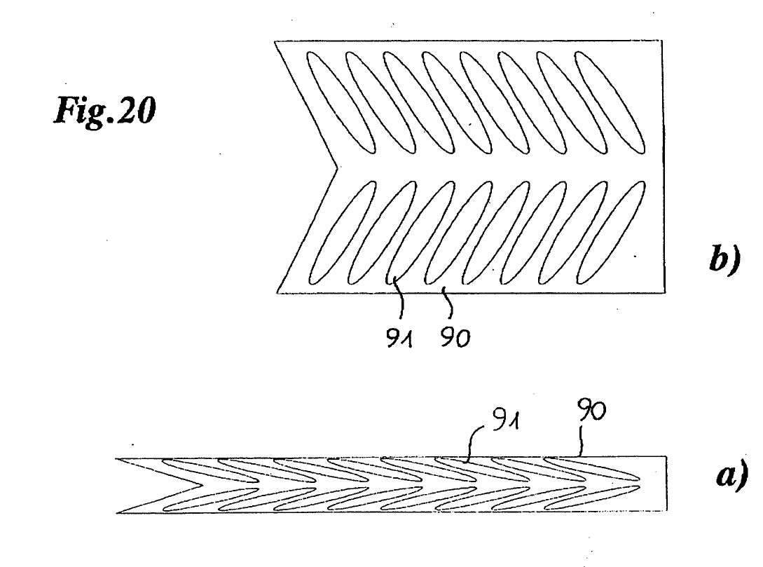

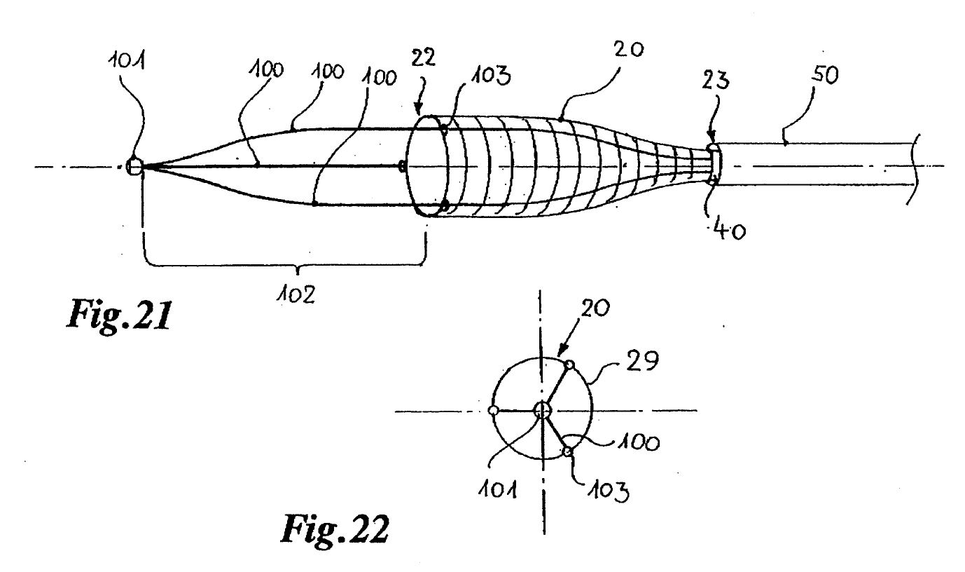

FIG. 21 shows a perspective view of an alternative embodiment of an extraction device according to the invention,

FIG. 22 shows a front view of the extraction device according to FIG. 21,

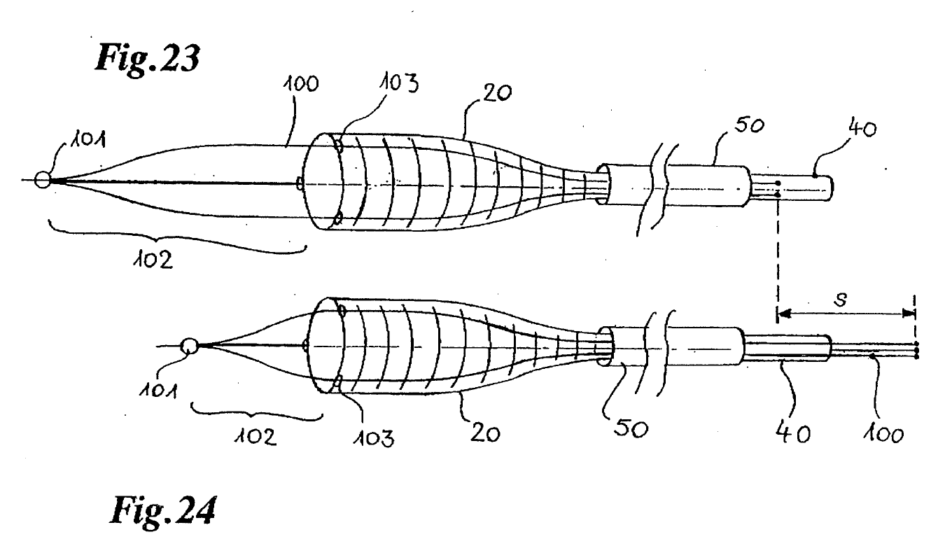

FIG. 23 and FIG. 24 show perspective side views of the extraction device according to FIG. 21 in a position of maximum projection from the channel element and in a position in which it has been partially drawn into said channel element,

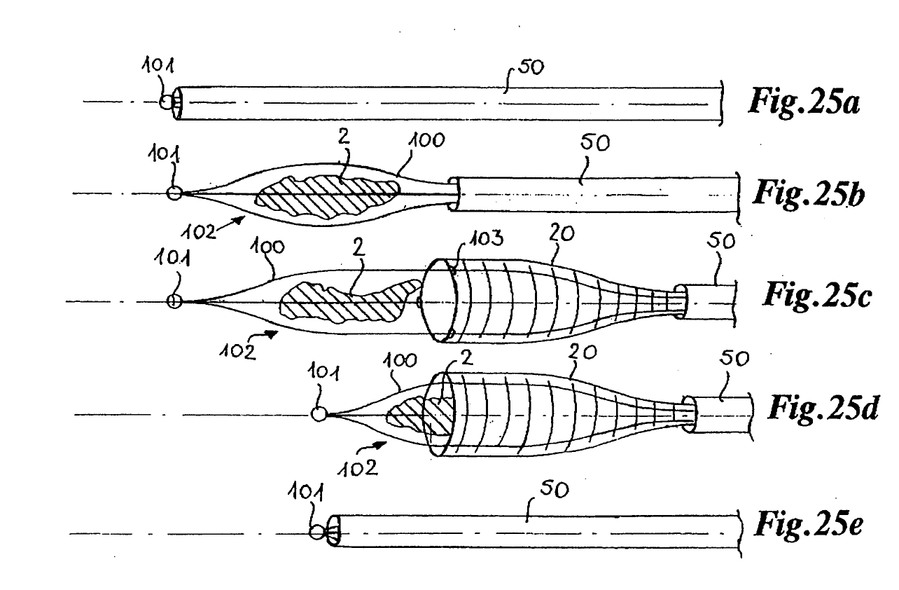

FIGS. 25a) through e) show individual steps in the process of recovering pulmonary emboli using an extraction device according to FIG. 21 and bringing them into a channel element,

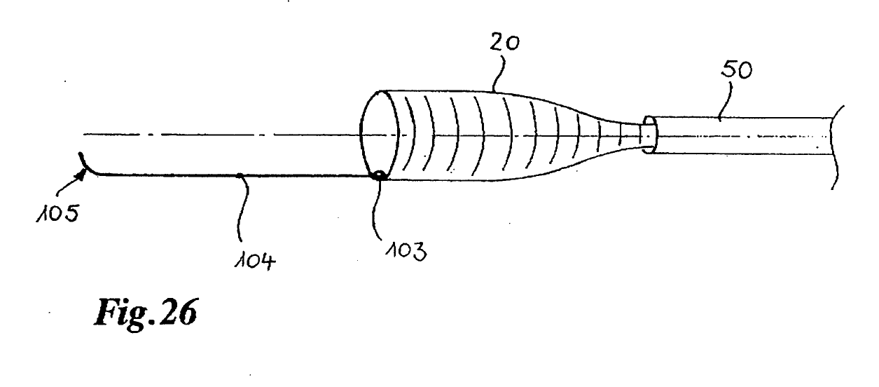

FIG. 26 shows a further embodiment of the extraction device according to FIG. 21 in which an eccentric guide wire is provided as a single-strut collecting basket,

FIG. 27 shows a further embodiment of an extraction device according to the invention with first and second collecting baskets,

FIG. 28 shows a further embodiment of an extraction device according to the invention with a coating applied to the mesh-like structure of the collecting basket,

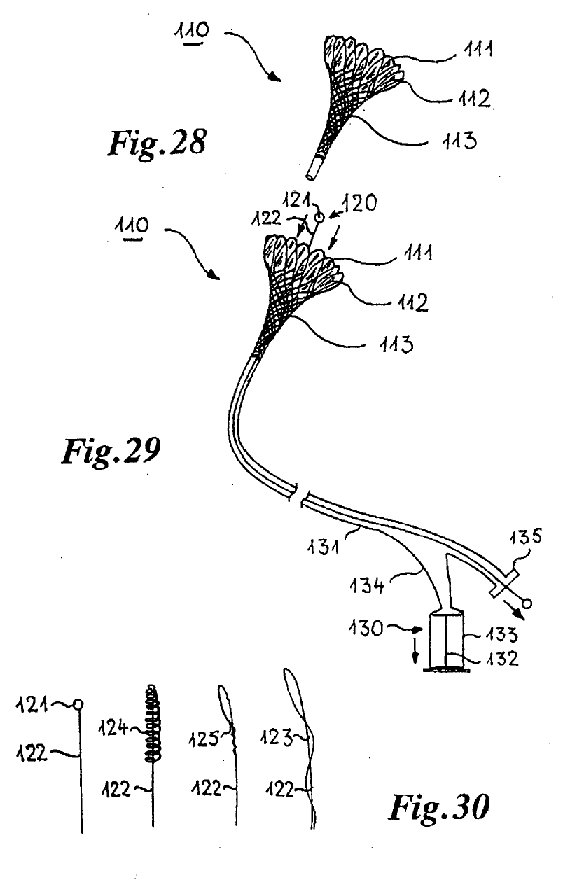

FIG. 29 shows a perspective view of the extraction device according to FIG. 28 provided with a separating means in the form of a wire with attached ball, and

FIG. 30 shows details of different embodiment variants of wire ends of a separating means.

FIG. 1 shows a first embodiment of an extraction device 1 according to the invention with a first collecting basket 10 and a second collecting basket 20.

Both collecting baskets are guided along a guide wire 30. They have adjustment elements 11, 21. The adjustment elements 11 of the first collecting basket are, like the guide wire 30, routed through the second collecting basket 20. The adjustment elements 11 and the guide wire are likewise routed through a catheter 40. All or some of the adjustment elements 11 can be guided displaceably or movably within said catheter.

The adjustment elements 21 of the second collecting basket are preferably routed outside the catheter in order to permit a better relative movement of the first and second collecting baskets and above all to permit adjustment of the second collecting basket. The second collecting basket is secured on the catheter 40, as can be seen from FIG. 2.

The first and second collecting baskets and the catheter 40 can be drawn into a channel element 50. The channel element 50 can itself be a catheter. It is pushed into a cavity of a human or animal body, in particular into a vessel, in order to bring the collecting baskets to an object 2 to be removed from the cavity 3. The object can, for example, be a clot, a foreign body, an embolus or another object which is lying in a vessel, in the pulmonary system, in the gastrointestinal system, the kidneys, the gallbladder or another part of the body or a cavity of a human or animal body and which is to be removed from there. To maintain the path through the body or cavities, the guide wire 30 is provided. This can have an atraumatic tip in order to avoid injury to the cavity, in particular the blood vessel.

The first collecting basket 10 has an umbrella-like design and comprises a distal closed end 12 and a proximal open end 13. Upon introduction into the channel element and in particular into the catheter, the collecting basket composed of a braided fabric is strongly compressed, whereas, after it has been pushed out from the channel element and the catheter, it expands so that its proximal end is able to receive the object that is to be removed.

By means of the adjustment elements 11, the first collecting basket can be positioned so that it is oriented optimally over the, object and pushes the latter in the direction of the second collecting basket after it has received said object. FIG. 1 shows the position where the first collecting basket has not yet received the object.

The second collecting basket 20 is shown in FIG. 1 as a. tubular elongate element. However, it can also have any other desired shapes, in particular the one shown in FIG. 2. In the latter, the proximal end 23 is secured in a sleeve element 24 and a tubular element 25. The distal end 22 of the second collecting basket is open, as in the embodiment according to FIG. 1. In the embodiment according to FIG. 2, the second collecting basket thus acquires a tulip shape. It is then possible, in particular by provision of adjustment elements, to ensure even better opening of the distal end of the second collecting basket for receiving the first collecting basket and the object to be recovered.

The second collecting basket is also made of a woven fabric. To permit better widening at least of the distal end 22 of the collecting basket 20, this woven fabric can be composed of filaments of different diameter. For example, a first filament 26 has a diameter of 0.20 mm and a second filament 27 has a diameter of 0.15 mm. However, any other desired diameters can also be chosen. These filaments of different diameter are intertwined, in particular alternately intertwined. In a partial area x, indicated in FIG. 2, it is possible simply to use a thinner filament and, in a second partial area, to use a thicker filament so that the first area can be widened better than the second area. This also makes it easier to receive objects in the distal area of the second collecting basket. It is also possible to use different filament diameter combinations in different areas.

FIG. 3 shows various possible ways in which the collecting baskets can be finished at the distal end and/or the proximal end. FIG. 3 shows only three different possibilities; there are, however, a great many others. Possibility a) indicates an interlacing of respectively contiguous filaments, as is customary in basket weaving. Variant b) indicates an interlacing with a smaller overhang at the edges, as is also customary in basket weaving. In variant c), eyelets are formed, as is advantageous in particular for applying adjustment elements to the first and second collecting baskets. Any desired combination of the three variants or of others can also be formed. FIG. 3 also indicates the different filament thicknesses.

A detail of the first collecting basket 10 is shown in FIG. 4. In this embodiment, the first collecting basket is secured on a guide cannula 14 via its distal end 12. The guide wire 30 is routed through the guide cannula. The adjustment elements 11 of the first collecting basket extend outside the guide cannula. The guide cannula, adjustment elements and guide wire are routed through the catheter 40. The latter in turn is arranged inside the channel element 50. In this embodiment, the second collecting basket is not present. The object can also be recovered without the second collecting basket.

In contrast to the embodiments of the first collecting basket according to FIG. 1 and FIG. 4, in the embodiment of the first collecting basket according to FIG. 5 the adjustment elements 11 are not secured separately on the collecting basket, but instead are made in one piece with the latter. Parts of the braided fabric of the first collecting basket are thus guided out and branched at the proximal end, with several filaments or wires of the braided fabric in each case being twisted together. This twisting together results in a plurality of adjustment element parts 15. These in turn are brought together in such a way that only two adjustment element parts 16 are routed to the catheter. The object 2 recovered in the first collecting basket 10 is located between the adjustment element parts 15.

In the embodiment according to FIG. 6, the adjustment element parts 15 are omitted and the individual ends of the braided fabric of the first collecting basket are brought together directly to form two adjustment element parts 16.

FIG. 7 shows an embodiment similar to FIG. 6, but in FIG. 7 some of the filaments 17 form loops at the proximal end and are routed back to the distal end, and only a few filaments form the adjustment element part 16.

In the embodiment according to FIG. 8, the filaments, in contrast to the other embodiments of the above-described first collecting baskets 10, are divided into two parts at an area distant from the proximal end of the collecting basket and these two parts are then guided together to form the two adjustment element parts 16.

In each embodiment of the first collecting baskets 10, when these are drawn into the catheter and/or into the channel element, the distance al of the distal end 12 from the proximal point of attachment 18 or point of emergence 19 remains substantially constant in order to permit simple introduction into the channel element or catheter. The point of attachment 18 here is intended to signify the point where the adjustment elements are attached to the first collecting basket, and the point of emergence is intended to signify the point where the adjustment elements formed in one piece with the collecting basket emerge from the braided fabric thereof.

FIG. 9 shows a further embodiment of a first collecting basket 10 designed according to the invention. In this embodiment, the filaments 17 at the proximal end are essentially interlaced in such a way that a straight finish is obtained. The ends of the filaments are guided together into the two adjustment element parts 16 in the area of the points of emergence 19 from the braided fabric. In this area, a reducing element 60 in the form of a noose is also provided. The noose is guided round the entire circumference of the distal end of the collecting basket 10. The noose element can preferably be actuated from outside of the patient's body. In this way it is possible to reduce the diameter of the opening at the proximal end of the collecting basket. An object recovered in the collecting basket can thus be better secured in the latter. At the distal end of the first collecting basket there is also a reducing element 61 which is likewise present in the form of a noose and is provided for reducing the diameter or for tying off this end. FIG. 9 also very clearly shows the formation of the distal end in respect of the interlacing of the individual filaments of the collecting basket. The distal end can thus be produced either by continuous intertwining or interlacing of the individual filaments or by tying off with a reducing element, as is indicated in FIG. 9. In FIG. 9, the adjustment element parts 16 are configured as tubular elements, which in particular can be made of plastic or metal.

FIG. 10 shows a further embodiment of a first collecting basket 10 designed according to the invention, with two adjustment elements 11, the collecting basket being asymmetrical, namely longer at one side. The lengthening piece 70 is made from the same braided fabric as the rest of the collecting basket. The lengthening piece 70 on one side is preferably drawn under the object to be recovered in such a way that the latter slides almost automatically into the opening 71 at the proximal end of the collecting basket. The lengthening piece 70 can be controlled using the two adjustment elements 11 secured at its proximal end 72. Instead of the two adjustment elements, it is also possible to use just one. At the proximal end 72 there is also a hook-shaped element 75 which can advantageously be engaged for releasing the collecting basket and for removing the collecting basket from the body of the patient. This embodiment of a collecting basket is especially suitable as a vena cava filter.

FIG. 11 shows a further embodiment of an extraction device 1 which is designed according to the invention and in which the collecting basket 10 is formed with its opening 73 directed away from the opening 28 of the second collecting basket 20. In this embodiment, the first collecting basket is once again secured on a guide cannula 74, but, in contrast to FIG. 1, via its proximal end 13. The distal end 12 is wide open in the view according to FIG. 11, so that an object to be recovered can be received therein.

The guide cannula 74 is routed through the second collecting basket and into the catheter 40 provided at the proximal end of the second collecting basket. With its proximal end 23, the second collecting basket is secured on the catheter 40 so as to be able to change its shape and position by way of the adjustment elements 21, that is to say be able to expand and compress it. The adjustment elements 21 are partially worked into the circumferential surface 29 of the second collecting basket or laced through the braided fabric of the circumferential surface. This permits particularly easy actuation of the second collecting basket. As is also shown in FIG. 11, the adjustment elements 21 of the second collecting basket are also preferably routed outside the catheter to its proximal end in order to permit actuation from outside the operating site. A channel element, through which the catheter 40, the guide cannula 74 and the adjustment elements 21 can be routed, is passed through the patient's skin and into the corresponding cavity to a point just before where the object to be collected lies. This, however, is not shown in FIG. 11.

The extraction procedure, or the capture of an object and its removal from the cavity, is performed by the channel element or a catheter, optionally with inner mandrel, being placed via a guide wire in the corresponding cavity, in particular a vessel. The catheter is positioned at a point over the object to be captured, in particular a clot, as far as its periphery. Then, the first collecting basket 10 is initially released. Thereafter, this is also done with the second collecting basket 20, but the latter is pushed out of the catheter before the clot and deployed. In this way, the clot is flanked on both sides by the collecting baskets. In the case of the embodiment according to FIG. 11, however, both collecting baskets are released before the clot so that the latter is located not only in front of the second collecting basket but also in front of the first collecting basket. After the collecting baskets have been released, the clot is captured using the first collecting basket and the latter is drawn back into the second collecting basket 20 together with the clot. For further recovery of the clot, both collecting baskets, in the state with one drawn into the other, are then drawn back into the channel element. Here, contraction of both collecting baskets takes place and, in this way, the clot is squeezed. This is done advantageously in the pulmonary artery or vena cava.

Another object, for example a foreign body or a body formed by the organism itself, such as a kidney stone or gallstone, is carried off in a corresponding manner, except that, depending on the consistency, the foreign body is not subjected to squeezing. It is simply recovered from the cavity and withdrawn to outside of the patient's body.

The guide wire is provided to maintain the approach route. To permit better control of the first collecting basket, the guide cannula can be used on this, as is shown in FIGS. 4 and 7 and FIG. 11. As can be seen in particular from FIG. 11, the adjustment elements, preferably wires, are secured at the ends, here the distal end 22 of the second collecting basket. In this way, it is possible to ensure that the second collecting basket can be optimally opened for drawing-in of the first collecting basket.

FIG. 12 shows a further embodiment variant for the first collecting basket 10. In contrast to the embodiment according to FIG. 11, this one is turned through 180°, in other words now points with its opening toward the distal opening of the second collecting basket. The guide cannula is in this way again secured at the distal end of the first collecting basket. This embodiment variant permits particularly effective capture of emboli, since these are often more widely scattered in a vessel, and this embodiment variant affords a comparatively widely opening collecting basket. The collecting basket shown in FIG. 12 can be obtained from the collecting basket 10 shown in FIG. 11 by turning this through 180°, e.g. also by pulling on adjustment elements which can be secured at the open end of the collecting basket.

In FIG. 13, the second collecting basket 20 is shown with its adjustment elements 21 and a catheter 40. The second collecting basket is shown substantially compressed, and the adjustment elements which contribute to the expansion are shown particularly clearly. Since it is the distal area x that is to be expanded for receiving the first collecting basket or the object to be recovered, the adjustment elements in this area are shown drawn out from the circumferential surface. In this way it can also clearly be seen that they are secured at the distal end 22 of the second collecting basket or laced through at this end, so that an adjustment element in each case adopts a forward and backward direction. The adjustment elements are guided outward at the proximal end 41 of the catheter. At this location, said adjustment elements can be actuated, in particular by a handgrip (not shown).

FIGS. 14 through 19 show different steps in the capture of an object, for example a foreign body in a vessel, by the second collecting basket 20. In FIG. 14, the elongate and unexpanded collecting basket is shown similarly to the one shown in FIG. 13. The distal end 22 of the second collecting basket is still substantially closed. By contrast, FIG. 15 shows an already opened distal end 22 of the collecting basket 20. The opening or expansion of the collecting basket in this distal area x is effected by the adjustment elements 21. In the upper part of the drawing in FIG. 15, the collecting basket is shown pushed out of the channel element 50 inside a vessel, and it has already been partially pushed over the object 2.

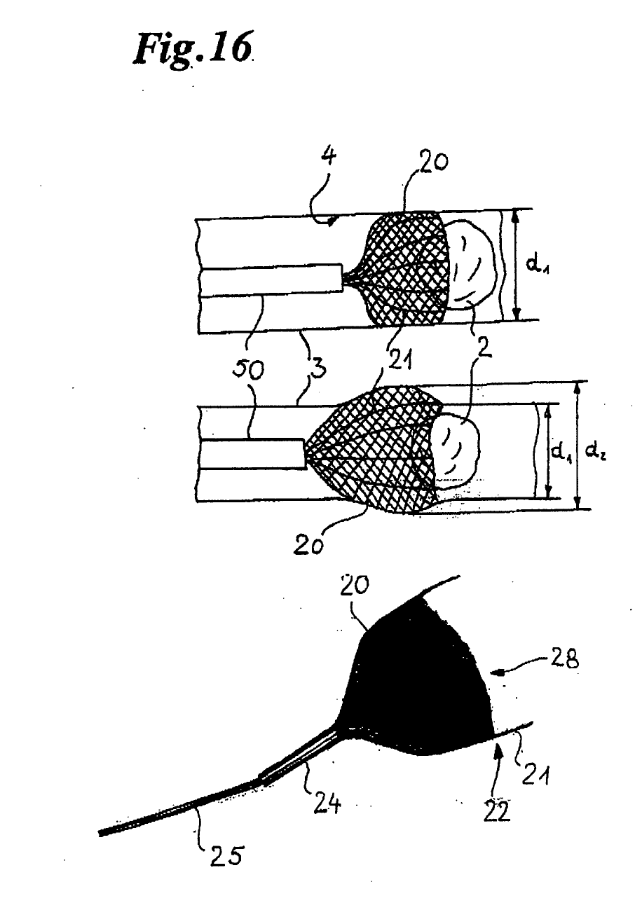

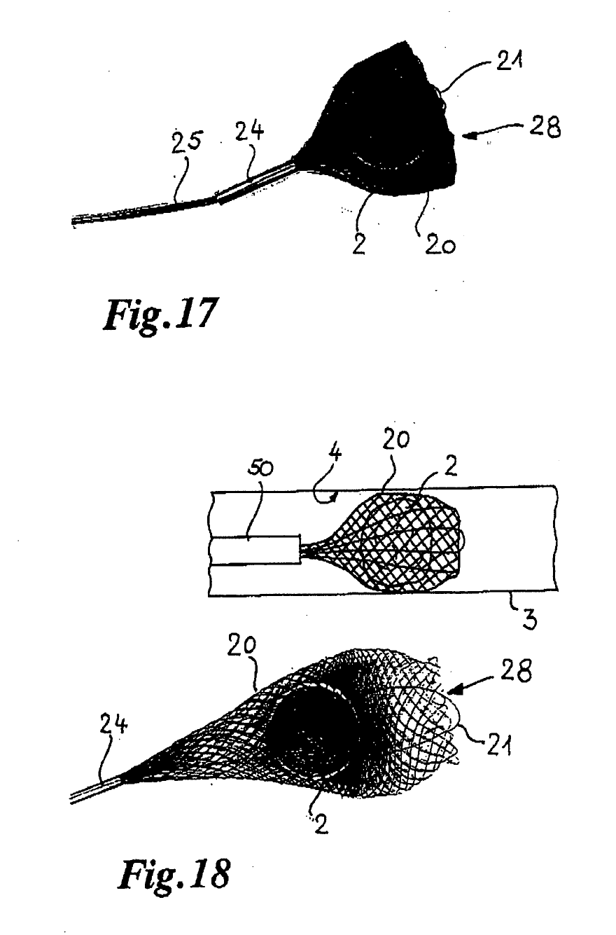

FIG. 16 shows the second collecting basket 20 now further expanded. In the topmost view, the collecting basket has been opened so wide that it bears completely on the inside wall 4 of the vessel. In the position indicated below this, the collecting basket, upon further expansion, partially opens the vessel further out, so that the vessel diameter di partially assumes a diameter d₂. By means of this widening of the vessel, the object 2 slides automatically into the opening 28 of the collecting basket. FIG. 17 shows the object once it has sprung or slid into the second collecting basket. Here, the collecting basket is shown still opened out at one end.

FIG. 18 shows a further step in capturing and recovering the object 2, in which step the adjustment elements 21 are released again. In this way, the tubular collecting basket draws together again in the distal area x, beginning in the area of the opening 28. In this way, the object 2 is prevented from sliding back out of the collecting basket. From the upper part of the drawing in FIG. 18 it will also be seen that, upon contraction of the collecting basket, the latter disengages again from the inside wall 4 of the vessel.

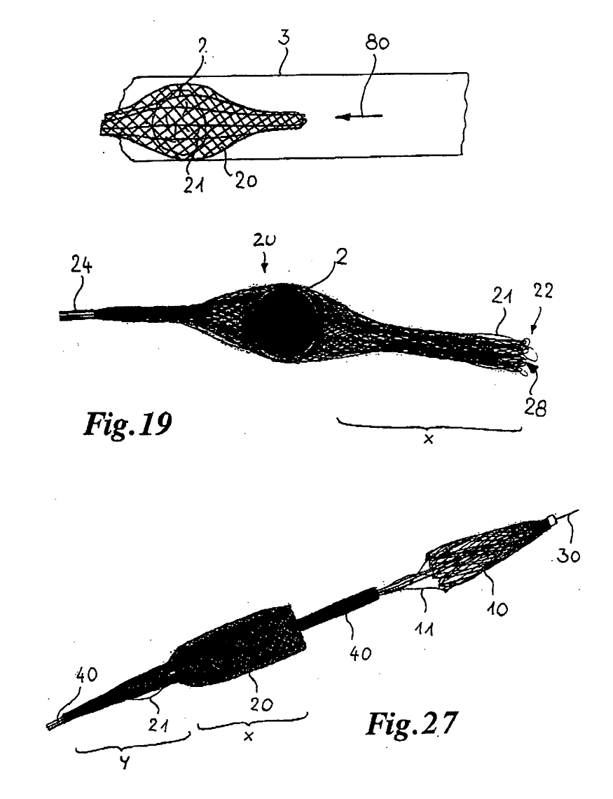

In the last step, which is shown in FIG. 19, the adjustment elements 21 are loosened so far that the woven fabric of the second collecting basket draws together again like a sock in the distal area x, so that the opening 28 is almost closed again at the distal end 22 of the collecting basket. The object 2 is received completely within the collecting basket. As will be seen from the upper part of FIG. 19, the collecting basket can now be drawn back out of the vessel or cavity, as is indicated by the arrow 80.

As can be seen in particular from FIGS. 15, 16 and 17, when the collecting basket opens out it also shortens. The more the collecting basket shortens, the more effective is its widening out and the better it is able to capture an object. This effect can be obtained not only with a wire braid or generally any braid of the collecting basket, but also by provision of a slotted tube 90, as can be seen from FIG. 20. The upper part of FIG. 20 shows the compressed slotted tube with cuts 91, whereas the lower part of FIG. 20 shows the expanded tube provided with cuts 91. In the embodiment shown, the arrangement of the cuts 91 is similar to the ribs of a skeleton, the individual cuts being arranged substantially parallel to one another. However, any other desired shape of cuts can also be chosen if it permits expansion and subsequent compression of the tube for changing its cross section. By providing particularly long cuts, a considerable change of cross section is made possible. The embodiment variant of a slotted tube shown in FIG. 20 is especially suitable for the second collecting basket. For the first collecting basket, a variant such as the one disclosed in DE 100 00 137 can be used, in particular the variant shown there in FIG. 1.

The present extraction device can also be used in conjunction with an endoscope. Endoscopy involves the provision of light sources, an optical device and a working channel. For use with an endoscope, the second collecting basket 20, for example, is clamped onto the first collecting basket 10 from outside. This can be obtained through the self-clamping force arising from the prestressing of the woven fabric or slotted tube of the second collecting basket. Independently of the use together with an endoscope, this self-clamping force is otherwise used in particular for holding the captured object. The first collecting basket 10 is subsequently introduced through the working channel. The second collecting basket 20 follows on from outside, owing to it being clamped to the first collecting basket. During the transport to the operating site, the second collecting basket clamped from outside holds secure. At the operating site, both collecting baskets are then opened. If appropriate, a channel element can be pushed over both collecting baskets and introduced into the working channel. In this mode of use, endoscopes with a diameter of 3 mm are especially suitable. Depending on the application, however, it is also possible to use larger endoscopes, for example endoscopes with a diameter of 10-15 mm. The way in which the object to be recovered is captured corresponds to the procedure already described above.

FIG. 27 shows a combination of some of the above-described embodiments of first collecting basket and second collecting basket. The second collecting basket 20 opens automatically in the distal area x. This part adapts and bears against the wall of the vessel or cavity. The proximal partial area y remains compressed or folded, but can be actively opened by pulling on the adjustment elements 21. After release of the adjustment elements 21, this proximal partial area is compressed again and, because of its imparted shape and shape-retaining force, securely holds the clot, emboli, etc.

FIGS. 21 and 22 show a further embodiment variant of the collecting basket 20. This is similar to the one shown in FIG. 2. The adjustment elements 100 used in this embodiment are guided out beyond the distal end 22 of the collecting basket 20. They are brought together at a distance from the distal end of the collecting basket 20 and are provided with an atraumatic ball 101. The portion of the adjustment elements 100 formed between the distal end of the second collecting basket 20 and the atraumatic ball 101 likewise serves as a collecting basket and is therefore designated by reference number 102. The collecting basket 102 is of open design, without any covering. The adjustment elements are guided through eyelets 103 at the distal end of the second collecting basket 20. At the proximal end of the collecting basket 20, the latter is secured in the catheter 40. Further covering can be provided by the channel element 50, as is set out in the preceding embodiments.

The collecting basket 20 can again be composed of a metal mesh or textile mesh, in which case the adjustment elements 100 and also the collecting basket 20 are made of an elastic self-expanding material. Here, for example, a metal wire can be used, in particular nitinol as shape-memory material.

By provision of the eyelets 103, the adjustment elements can be shortened relative to the collecting basket 20 in order to draw a captured object, in particular embolism material, into the collecting basket 20. By pushing the channel element 50 forward, the closed collecting basket 20 and the open collecting basket 102 are radially compressed and thereafter completely covered by the channel element. Conversely, upon withdrawal of the catheter, both collecting baskets expand, because of their marked elasticity and shape, to their maximum diameter of deployment. This corresponds to the embodiments of collecting baskets shown in the preceding figures. To capture embolism material, or generally an object, it is not absolutely necessary to draw the open collecting basket 102 into the closed collecting basket 20. Instead, the closed collecting basket 20 can be lengthened or pushed forward in relation to the open collecting basket 102 so that the object captured in the open collecting basket again ends up in the closed collecting basket 20.

It is possible to provide a relatively small number of adjustment elements 100, in particular just three or four, these serving as guide wires for the advance movement of the collecting basket 20 in the deployed state. Moreover, a small number of adjustment elements means that the capture of an object is not impeded.

The front view in FIG. 22 shows that all three adjustment elements are arranged at a uniform distance from one another, namely at an angle of approximately 120°, on the circumferential surface of the collecting basket 20. However, a non-uniform distribution across this circumference is also possible.

FIGS. 23 and 24 show the relative adjustment of the open collecting basket 102 with respect to the closed collecting basket 20. This view also shows the catheter 40 inside the channel element 50, with which catheter the collecting basket 20 is securely connected at its proximal end. Instead of eyelets 103, other guide elements can also be used. These serve in particular to stabilize the coaxial movement of the collecting basket 102 relative to the collecting basket 20, and vice versa. By this means, a purely axial movement is allowed, whereas a radial movement or a movement tangential to the circumference is largely avoided.

The distance s by which the adjustment elements 100 are drawn out at the proximal end of the catheter corresponds substantially to the distance by which the open collecting basket 102 is drawn into the closed collecting basket 20 and by which the open collecting basket thus shortens.

FIG. 25 shows a sequence of the procedure of capturing an object 2 using the embodiment of an extraction device according to the invention shown in FIGS. 21 through 24. Here, the channel element 50 with an inner mandrel is first introduced over a guide wire into, for example, a pulmonary artery circulation. After removal of the inner mandrel and, if appropriate, of the guide wire, the open collecting basket 102 is first pushed out of the channel element until the atraumatic ball 101 emerges from the distal end of the channel element (see FIG. 25a)). After the channel element has been drawn back, the open collecting basket 102 is released. The adjustment elements 100 expand and can be positioned around the object to be captured, as is represented in FIG. 25b). During the capturing procedure, the whole extraction device can be rotated and the capturing thus made easier. Upon further withdrawal of the channel element 50, the collecting basket 20 is also released, as is shown in FIG. 25c). The open collecting basket 102 is then drawn back into the closed collecting basket 20 and, in so doing, is shortened, the captured object being drawn into the collecting basket 20, as is shown in FIG. 25d). In the fifth step shown in FIG. 25 (see FIG. 25e)), the channel element is pushed distally in relation to the collecting basket 20 and, in this way, both collecting baskets are drawn completely into the catheter and compressed therein. During this procedure, the liquid portion of the captured object, in particular of a clot or embolus, can be pressed through the meshes of the covering of the closed collecting basket 20. The cell and tissue matrix of the clot or embolus then remain in the inside of the collecting basket. This matrix can be extracted outside the patient's body by drawing back only the catheter or channel element. After cleaning the two collecting baskets and reintroducing both of them over the channel element still lying in the pulmonary artery, the procedure can be repeated until the pulmonary artery is recanalized.

In an alternative embodiment, it is also possible for the adjustment elements 100 to run inside the circumferential surface 29 of the collecting basket 20. This means it is possible to use a particularly large collecting basket 20 which can bear completely against an inside wall of a vessel without externally routed adjustment elements 100 or eyelets damaging this. An interlacing of the adjustment elements with the circumferential surface 29 is also possible.

In a further embodiment, which is shown in FIG. 26, instead of three adjustment elements 100, only one adjustment element 104 is provided. The latter is again routed through an eyelet 103, but lies on the inside of the collecting basket 20. The adjustment element 104 is also axially displaceable and assumes the function of a guide wire. The adjustment element is curved at its distal end 105 in order to be able to take hold of objects to be captured. For this purpose, the distal end 105 can also be spoon-shaped, for example, to make this holding easier.

The adjustment elements, and in particular also the one adjustment element 104, continue along the circumferential surface of the collecting basket 20 in the axis of the catheter or channel element, at the same time permitting a relative axial movement.

FIG. 28 shows a further embodiment of a collecting basket 110 according to the invention. In contrast, for example, to the collecting baskets shown in FIGS. 17 and 18, the collecting basket 110 has a coating 111. This is provided in the interstices 112 of the mesh-like structure 113 of the collecting basket. It is applied, for example, by immersion of the mesh-like structure of the collecting basket. This coating is designed as a membrane-like structure of the collecting basket or as a film within the mesh-like structure of the collecting basket. The membrane-like structure is suitably obtained using film-forming materials such as, for example, a natural or synthetic polymer formed from one or more monomers, in particular formed by polyaddition, polymerization or polycondensation, in particular a polycarbonate, polyester, polyamide, polyolefin or polyurethane. Polystyrenes too are suitable for the coating. Depending on the application, a material can be chosen having a greater or lesser flexibility or surface tension. It should in any event be so flexible that a capturing procedure is not impeded by the coating but instead even improved by the latter. The provision of such a coating is expedient in particular for the capture of very small objects or object parts so that, when recovered, they are not lost from the collecting basket. A coating can be provided in each one of the above-described embodiments of collecting baskets.

All the above-described embodiments of collecting baskets can have a coating of the individual elements of their structure, in particular of the wires from which they are formed. Such a coating can suitably be a bioactive surface coating, a coating with heparin, a carbonization of nitinol when nitinol is used as shape-memory material for forming the at least one collecting basket, nanotechnological or biotechnological coatings, the application of radiopaque particles, in particular tantalum particles, a coating releasing an active substance, e.g. a coating with polymers, in particular polyurethane, which is/are impregnated with active substances, e.g. medicaments. Such provision of medicaments and other active substances proves especially advantageous when the extraction device is used as a vena cava filter. The polymers can also be microporous. By means of such a coating of the wires or elements of the structure of the collecting basket, it is possible to avoid the formation of (new) clots and also the adherence of endothelial cells. The material chosen for the coating is preferably one that does not cause inflammation in the patient's body and does not lead to formation of clots (=magic hat coating).

If the captured clot or captured material is too large to be able to be drawn into a catheter, a means 120 is advantageously provided for cutting up the clot in the area of the collecting basket (see FIG. 29). Such a means 120 can, for example, be a wire 122 which is provided with a ball 121 or similar means and which is arranged inside the collecting basket, in particular the collecting basket according to FIG. 28 closed from outside by provision of the coating, and can be drawn back into the latter and in so doing cut up the clot or captured object. Instead of a wire provided with a ball, any other desired means of cutting up the clot or captured object can be provided, for example an additional wire noose 123, a coiled portion 124, a noose-shaped portion 125, a combination of different thicknesses or the like. Four different embodiment variants are shown in FIG. 30.

For introduction of the collecting basket comprising a wire provided with a material thickening, a guide wire is first pushed through a catheter to the site in the patient's body from where the object, in particular the clot, is to be removed. A channel element is thereafter advanced over the guide wire, and the guide wire is then removed. The material thickening, in particular in the form of a ball, is advanced on a newly introduced wire or corresponding element via the channel element as far as the object and cuts the latter up. If the distal portion of a wire or similar element is provided with a coiled or noose-shaped portion, this wire or this element can itself be used as guide wire. This dispenses with the need for preliminary insertion of a guide wire.

Instead of using a wire, it is likewise possible to use a balloon catheter together with a stent or similar expandable tubular element. Such an embodiment is particularly suitable for use in the case of arteriosclerosis, for example, for removing, as foreign body, the calcium material deposited on the vessel wall. For example, the balloon catheter together with the collecting basket according to FIG. 28 is introduced into the patient's vessel, for example the carotid artery, the collecting basket is opened, and the foreign body captured and removed. The balloon catheter permits widening of the vessel and thus release of foreign bodies adhering to the wall.

To make it easier to transport the captured and cut up clot fragments and objects back out, a means 130 can advantageously be provided for suctioning the clot fragments or object parts and objects. Such a suction means comprises, for example, a cannula 131 which ends in the area of the collecting basket and via which the clot fragments or object parts and objects can be sucked out. For this purpose, a partial vacuum is generated at the other end of the cannula, for example via a plunger 132 in a cylinder part 133. The cannula can advantageously be brought by a catheter to the site in the body of the human or animal from where the clot or object is to be removed. Such a suction means can be advantageously used both in the embodiments with normal mesh-like structure and also in the coated embodiment according to FIG. 28, as is shown in FIG. 29, and above all also where a means is provided for cutting up or separating the object or object parts. Toward the proximal end, the cannula 131 has a branch piece 134 comprising the cylinder part 133 with plunger 132 and a grip part 135 for holding when actuating the wire 122. The arrows in FIG. 29 indicate the direction of withdrawal and the direction in which a partial vacuum is generated for sucking objects out.

In addition to the embodiments mentioned and described above, a great many others are possible, in particular also combinations of the described forms in which, where first and second compressible and expandable collecting baskets are provided between which objects can be captured, the collecting baskets being able to be drawn one into the other, at least one collecting basket has an umbrella-like design in the expanded state. With provision of at least one compressible and expandable collecting basket having a distal end and a proximal end, at least one adjustment element can be secured at the distal and/or proximal end in such a way that the at least one collecting basket can be controlled by the latter and changed in shape. In addition, or alternatively to this, a coating of the collecting basket is also possible. In particular, it is also possible to use combinations of sectioned tubes and braided fabrics, scrims or woven fabrics with suitable choice of material for the collecting basket or baskets.

### LIST OF REFERENCE LABELS

1 extraction device
2 object
3 cavity
4 inside wall of vessel
10 first collecting basket
11 adjustment element
12 distal end
13 proximal end
14 guide cannula
15 adjustment element part
16 adjustment element part
17 filament
18 point of attachment
19 point of emergence
20 second collecting basket
21 adjustment element
22 distal end
23 proximal end
24 sleeve element
25 tubular element
26 first filament
27 second filament
28 opening
29 circumferential surface
30 guide wire
40 catheter
41 proximal end
50 channel element
60 reducing element
61 reducing element
70 lengthening piece
71 opening
72 proximal end
73 opening
74 guide cannula
75 hook-shaped element
80 arrow
90 slotted tube
91 cut
100 adjustment element
101 atraumatic ball
102 collecting basket
103 eyelets
104 adjustment element
105 distal end
110 collecting basket
111 coating
112 interstice
113 mesh-like structure
120 means for cutting
121 ball
122 wire
123 wire noose
124 helical portion
125 noose-shaped portion
130 suction means
131 cannula
132 plunger
133 cylinder part
134 branch piece
135 grip part
x distal area
y proximal partial area
a₁ distance
d₁ vessel diameter
d₂ widened vessel diameter
s stretch

1. An extraction device for extracting objects, in particular clots, foreign bodies, etc., from cavities in a human or animal body, with first and second compressible and expandable collecting baskets between which the object can be captured, said collecting baskets being mutually displaceable and being able to be drawn one into the other, wherein at least one collecting basket is umbrella-like in the expanded state and is designed with flexible wire-like adjustment elements for deliberately changing the shape and/or position of the collecting basket so that the object can be captured in the latter and can be drawn into the other collecting basket.

2. The extraction device as claimed in claim 1, wherein the umbrella-like collecting basket opens in the direction toward the other collecting basket or in the direction away from the other collecting basket.

3. The extraction device as claimed in claim 1, wherein both collecting baskets are provided with at least one adjustment element for deliberately adjusting the shape and/or position of the collecting baskets.

4. An extraction device for extracting objects, in particular clots, foreign bodies, etc., from cavities in a human or animal body, with at least one compressible and expandable collecting basket having a distal end and a proximal end, wherein at least one wire-like flexible adjustment element is secured at the distal and/or proximal end in such a way that the at least one collecting basket can be deliberately oriented by the latter and changed in shape.

5. The extraction device (o as claimed in claim 4, wherein the at least one adjustment element has one or more thin wires.

6. The extraction device as claimed in claim 4, wherein the at least one adjustment element is arranged on the outside and/or inside of the at least one collecting basket, in particular at least partially integrated into the circumferential surface of the collecting basket and/or laced into this.

7. The extraction device as claimed in claim 4, wherein the at least one adjustment element protrudes beyond the outstretched length of the at least one collecting basket and is arranged to be actuated in particular from the proximal end, in particular to be actuated via a handgrip.

8. The extraction device as claimed in claim 4, wherein, with an adjustment element provided at the proximal end of the collecting basket, the latter has an asymmetrical design, in particular lengthened on one side in the area of attachment of the adjustment element, and/or is provided with a hook-shaped element for engagement of an adjustment and/or guide element.

9. The extraction device as claimed in claim 4, wherein the adjustment element or elements are secured on the collecting basket in a branched-out configuration and are brought together in groups proximally.

10. The extraction device as claimed in claim 4, wherein the at least one adjustment element is in one piece with the collecting basket.

11. The extraction device as claimed in claim 4, wherein the distance between the distal end of the collecting basket and at least one proximal point of attachment or point of emergence of the at least one adjustment element is constant for different designs of the collecting basket.

12. The extraction device as claimed in claim 4, wherein the proximal end of the at least one collecting basket can be fixed or is fixed in a tubular element, in particular a catheter, and the adjustment element or elements are guided or can be guided through the tubular element.

13. The extraction device as claimed in claim 4, wherein the at least one collecting basket is designed so that it shortens in its longitudinal direction upon widening and lengthens when its cross section is reduced, and in particular can be expanded to a diameterd, greater than the diameter of the cavity to be cleared, for partial widening of the cavity.

14. The extraction device as claimed in claim 12, wherein a sleeve element for strengthening the connection between tubular element and collecting basket is provided at the proximal end of the at least one collecting basket.

15. The extraction device as claimed in claim 12, wherein the tubular element is made in one piece with the second collecting basket and is provided at least partially with a cut permitting expansion and compression.

16. The extraction device as claimed in claim 11, wherein reducing elements arranged transversely with respect to the longitudinal extent of the at least one collecting basket are provided, in particular in the area of the proximal and/or distal ends of the collecting basket and/or in the area of the at least one proximal point of attachment or point of emergence of the at least one adjustment element, and the reducing elements are in particular nooses.

17. The extraction device as claimed in claim 4, wherein the adjustment element or elements are fixed or movably guided in at least one tubular element, in particular a catheter.

18. The extraction device as claimed in claim 1, wherein a guide cannula is provided which is secured on the distal end of the first collecting basket.

19. The extraction device as claimed in claim 18, wherein the guide cannula and/or the tubular element or elements is/are made of a flexible material, in particular a metal, a metal alloy, a plastic or another flexible material or a combination of materials, in particular of nitinol.

20. The extraction device as claimed in claim 4, wherein a channel element is provided which has an internal diameter such that the at least one collecting basket, a guide cannula and/or tubular elements and the adjustment element or elements can be guided through it.

21. The extraction device as claimed in claim 20, wherein the channel element is made of a stable and at least partially flexible material, in particular of a plastic, metal, a metal alloy, in particular nitinol, in particular a thin-walled nitinol tube.

22. The extraction device as claimed in claim 4, wherein at least one collecting basket is made of a braided fabric and/or woven fabric and/or scrim, in particular a wire braid and/or woven wire fabric and/or wire scrim and/or at least one collecting basket is composed of a tube slotted along at least part of its length and/or is provided with a coating.

23. The extraction device as claimed in claim 4, wherein the at least one adjustment element is formed from a part of a braided fabric, woven fabric, scrim, or a slotted tube.

24. The extraction device as claimed in claim 22, wherein the cuts in the slotted tube are made in such a way as to afford the maximum ratio of shortening and widening upon expansion of the collecting basket.

25. The extraction device as claimed in claim 24, wherein the cut or cuts in the slotted tube are made long in comparison to the lengthwise extent of the collecting basket.

26. The extraction device as claimed in claim 4, wherein the at least one collecting basket is made of a biocompatible material, in particular a metal or a metal alloy, in particular a stainless steel or nitinol and/or the material of the at least one collecting basket is coated with a material, in particular a biocompatible surface coating, heparin, a carbonization of nitinol, a nanotechnological coating, radiopaque particles, a coating releasing active substance, an in particular microporous biotechnological or other coating.

27. The extraction device as claimed in claim 4, wherein partial areas of the at least one collecting basket are made of material of different diameter, in particular an expandable partial area (x) of the at least one collecting basket is made of a material with a thinner cross section or has a braided fabric or scrim or woven fabric with filaments of different diameter.

28. The extraction device as claimed in claim 27, wherein the material of the at least one collecting basket in at least one partial area is chemically and/or mechanically treated, in particular etched, electrolytically polished, microground or otherwise treated.

29. The extraction device as claimed in claim 4, wherein a guide wire and/or inner mandrel is provided along which the at least one collecting basket can be displaced and/or can be inserted into the cavity.

30. The extraction device as claimed in claim 1, wherein at least the second collecting basket has a self-opening partial area (x) and a self-closing partial area (y), which self-closing partial area (y) can be opened deliberately by at least one adjustment element.

31. The extraction device as claimed in claim 4, wherein the extraction device can be used in conjunction with an endoscope with or without provision of a channel element.

32. The extraction device as claimed in claim 4, wherein a means is provided for cutting or separating objects, in particular a wire provided with a material thickening, in particular a ball, a helical portion, a noose-shaped portion, a combination of these or some other type of material thickening, which wire can be or is arranged so as to be movable inside the collecting basket, and/or a balloon catheter provided with a stent or such like element.

33. The extraction device as claimed in claim 4, wherein a suction means is provided for suctioning of objects or parts of objects, in particular a cannula or such like tubular means which can be guided into the area of the collecting basket and can be acted on by a partial vacuum.
